(19) United States
(12) Patent Application Publication
(10) Pub. No.: US 2012/0089089 A1
(43) Pub. Date: Apr. 12, 2012

(54) METHODS OF MAGNETICALLY GUIDING AND AXIALLY ALIGNING DISTAL ENDS OF SURGICAL DEVICES

(75) Inventors: Christopher P. Swain, London (GB); William D. Fox, New Richmond, OH (US)

(73) Assignee: Ethicon Endo-Surgery, Inc., Cincinnati, OH (US)

(21) Appl. No.: 12/902,550

(22) Filed: Oct. 12, 2010

(51) Int. Cl. A61M 25/092

(52) U.S. Cl. 604/95.01

(57) ABSTRACT

A magnetic alignment and guidance system is described for use during a surgical or diagnostic procedure within a patient to align and magnetically join the free ends of catheters to form a continuous channel. The system includes two magnetic couplers mounted on the ends of the catheters. One or both of the catheters may be attached to a surgical or diagnostic device, such as a trocar or an endoscope.

### BACKGROUND

i. Field of the Invention

The present application relates to methods and devices for use in minimally invasive surgical procedures and, more particularly, to methods and devices for aligning and magnetically joining medical devices in vivo.

ii. Description of the Related Art

In minimally invasive surgical procedures, such as laparoscopic surgery, a surgeon may place one or more small ports into a patient's abdomen to gain access into the abdominal cavity of the patient. A surgeon may use, for example, a port for insufflating the abdominal cavity to create space, a port for introducing a laparoscope for viewing, and a number of other ports for introducing surgical instruments for operating on tissue. Other minimally invasive surgical procedures include natural orifice transluminal endoscopic surgery (NOTES™) wherein surgical instruments and viewing devices are introduced into a patient's body through, for example, the mouth, nose, vagina, or rectum. The benefits of minimally invasive procedures compared to open surgery procedures for treating certain types of wounds and diseases are now well-known to include faster recovery time and less pain for the patient, better outcomes, and lower overall costs.

The foregoing discussion is intended only to illustrate various aspects of the related art in the field of the invention at the time, and should not be taken as a disavowal of claim scope.

### SUMMARY

An alignment and guidance system is described. The system includes a pair of magnetic couplers comprising a first and a second coupler. The first coupler has a free magnetic end that defines a first magnetic field around the free end of the first coupler. The first coupler also has an attachable end and defines a channel therethrough. The attachable end is structured for attachment to an open end of a first catheter. The second coupler has a free magnetic end that defines a second magnetic field around the free end of the second coupler. The second coupler has an attachable end and defines a channel therethrough. The attachable end is structured for attachment to an open end of a second catheter. The free magnetic end of the second coupler is magnetically attracted to the free magnetic end of the first coupler such that upon contact between the first and second magnetic fields, the first and second free ends couple to define a continuous channel from through the first and second couplers.

When the attachable ends are attached to first and second catheters, the continuous channel is defined from the first catheter to the second catheter. In certain embodiments, one or both of the catheters are attached to a surgical instrument, such as a trocar or an endoscope. In certain embodiments, one or both of the catheters either join or extend through, ports in different surgical instruments.

The alignment and guidance system may include embodiments wherein the first and second couplers are fixed to the ends of the first and second catheters, respectively. In other embodiments, the first and second couplers are releasably attached to the first and second catheters, respectively.

A method of aligning the channels of opposing catheters in an internal site within a patient during a surgical or diagnostic procedure is also described herein. The method includes the steps of attaching one of the couplers described herein to a free end of each of two catheters, directing one catheter from a first entry point of the patient to an internal site within the patient with the free magnetic end of one of the couplers positioned at the leading distal end of one of the catheters, and directing the other catheter from a second entry point of the patient to the internal site within the patient with the free end of the other coupler positioned at the leading distal end of the other catheter. The magnetic fields of the free ends of each coupler are brought into contact to thereby attract the free ends of each coupler to the other and align the channels of each coupler. Thereafter, an instrument may be inserted through the channel of one of the catheters through the free end of the coupler attached thereto into the free end of the other coupler, and where both catheters have channels, into the channel of the opposing catheter.

The instrument may be a guide wire, a third catheter, or a tether, an electrical wire, tubing for carrying fluid into or out of the patient, and the like.

In one embodiment, the instrument may be a blade for making an incision in a band or wall of tissue separating the free ends of the couplers. The method may further provide, after the incision is made, passing one of the couplers or the coupler and catheter through the incision, and coupling the free ends of the couplers to define a continuous passage through the channels of each catheter and each coupler.

The method may further include withdrawing the blade from the channel of the catheter and passing a second instrument through the channel of one of the catheters through the continuous passage. The second instrument may be a guide wire, a third catheter, or a tether, an electrical wire, tubing for carrying fluid into or out of the patient, and the like.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 shows a perspective view of two magnetic couplers, each attached to an end of a catheter associated with a surgical or diagnostic device.

FIG. 2 shows a sectional view of the two magnetic couplers of FIG. 1 with a needle or guide wire passing through the channel of each coupler and associated catheter and through a band of the patient's tissue.

FIG. 3 shows one magnetic coupler extending from one port in the end of a surgical instrument.

FIG. 4 shows a sectional view of the two couplers of FIG. 1 magnetically joined to define a single continuous channel and a needle or guide wire passing through the channel.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

As used herein, the term “biocompatible” includes any material that is compatible with the living tissues and system(s) of a patient by not being substantially toxic or injurious and not causing immunological rejection. “Biocompatibility” includes the tendency of a material to be biocompatible.

As used herein, the term “longitudinal axis”, with respect to an instrument, means the exact or approximate central axis defined by said instrument along its greater dimension, i.e., along its length, from its distal end to its proximal end, and vice versa, and is not intended to be limited to imply a straight line, wherein, for example, an instrument includes a bend angle as described herein, it is intended that “longitudinal axis” as used herein follows such bend angle. As used herein, the term “axial” or “axial movement” or variants thereof, with respect to an instrument or a component of an instrument, means the movement in the direction of the longitudinal axis of such instrument.

As used herein, the term “patient,” used herein, refers to any human or animal on which a suturing procedure may be performed. As used herein, the term “internal site” of a patient means a lumen, body cavity or other location in a patient's body including, without limitation, sites accessible through natural orifices or through incisions.

During some surgical and diagnostic procedures, especially NOTES procedures, where one may need to pass guide wires from the surgical patient's peritoneum into the GI tract and perhaps out of the patient's mouth, or in the reverse order, it may be necessary or desirable to align the ends of two devices to form a continuous lumen between the two. It may also be necessary to align the ends of the devices to simultaneously pierce an internal tissue barrier between the devices, either as a location guide or to form the continuous lumen.

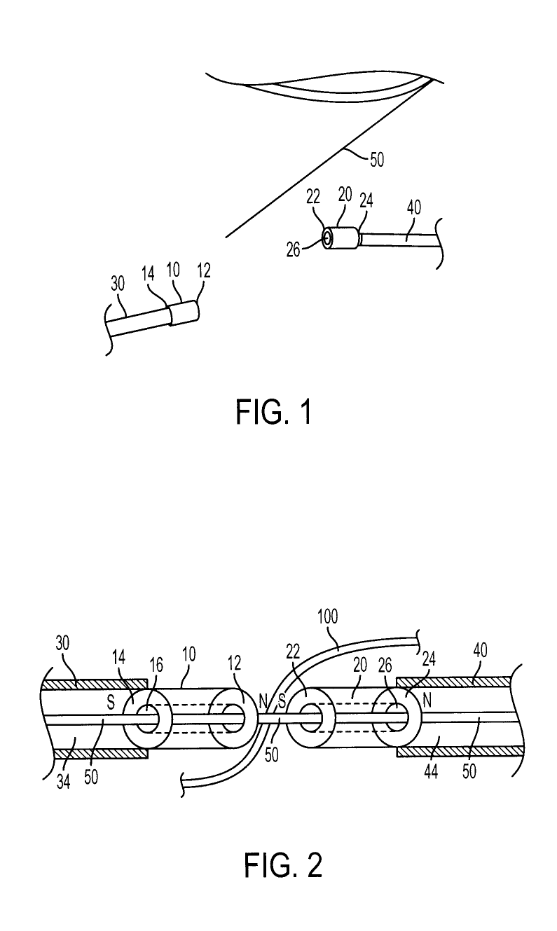

Referring to FIG. 1, an embodiment of a guidance and alignment system includes a pair of magnetic couplers 10 and 20, each mounted on an end 32 and 42, respectively, of a different one of the two catheters 30 and 40.

As shown in FIG. 2, magnetic coupler 10 is a hollow tubular member defining a channel 16 and having an end 14 attached to a free end 32 of catheter 30. The end 14 may be attached to catheter 30 by any suitable means known in the art, such as by mating threaded ends, or other complementary mating structures on each of the ends 14 and 32.

Coupler 10 may be made of any biocompatible material, but has at least one free magnetic end 12 made in a preferred embodiment of a ferromagnetic material. In one embodiment, end 12 of coupler 10 is monopolar. Alternatively, all of the coupler 10 may be made of a biocompatible ferromagnetic material. In certain embodiments, coupler 10 is a dipole and free end 12 has the polarity of “magnetic north” and attachable end 14 is also magnetic and has a polarity opposite that of the free end 12, i.e., “magnetic south”. Those skilled in the art will recognize that the polarity of the ends 12 and 14 may be the opposite so that free end exhibits “magnetic south” and attached end 14 exhibits “magnetic north”. In any of the various embodiments, a magnetic field is defined around the free end 12,

Coupler 20 is also a hollow tubular member defining a channel 26 and may be made of any biocompatible material. Like coupler 10, coupler 20 has at least one free magnetic end 22, made in certain embodiments of a ferromagnetic material, which, in one embodiment, has the polarity of “magnetic south” and defines a magnetic field around the end 22. Coupler 20 has an attachable end 24 for attachment to catheter 40. Attachable end 24 may be attached to catheter 40 by any suitable means known in the art, such as by mating threaded ends, or other complementary mating structures on each of the ends 24 and 42. In various embodiments, all of the coupler 20 may be made of a biocompatible ferromagnetic material wherein attachable end 24, like free end 22, is magnetic and has a polarity opposite that of the free end 22, i.e., “magnetic north.” Those skilled in the art will recognize that the polarity of the ends 22 and 24 may be the opposite (i.e., one end “north” and the other end “south”), provided that the magnetic ends 12 and 22 are of opposite polarity so that they magnetically attract each other.

Suitable ferromagnetic materials include nickel, iron, cobalt, gadolinium and their alloys, such as stainless steel or another suitable biocompatible steel alloy. In some embodiments, where an external magnetic field can be applied, paramagnetic materials may be used for ends 12 and 22. Electromagnets may be used in certain embodiments where an electric current can be applied. A source of electrical power would be provided by an electrical tether within or running alongside catheters 30, 40.

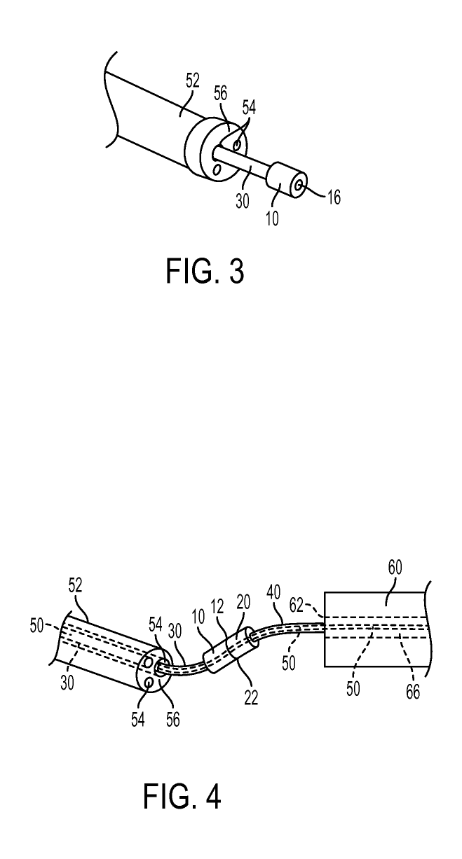

Catheter 30 defines a channel 34 and catheter 40 defines a channel 44. Catheter 30 with its corresponding magnetic coupler 10 mounted thereon, may be operatively connected to the distal end 56 of a therapeutic, surgical or diagnostic device 52, such as a laparoscopic trocar, endoscope or the like, or may be passed through a port 54 of any such device to extend from the distal end 56 thereof, as shown in FIG. 3.

As used herein, “surgical device” shall refer to any such therapeutic, surgical or diagnostic device, such as a laparoscopic trocar, endoscope or the like, to which a catheter may be operatively connected or through which a catheter may pass.

As used herein, the term “operatively connected” with respect to two or more components, means that operation of, movement of, or some action of one component brings about, directly or indirectly, an operation, movement or reaction in the other component or components. Components that are operatively connected may be directly connected, may be indirectly connected to each other with one or more additional components interposed between the two, or may not be connected at all, but within a position such that the operation of, movement of or action of one component effects an operation, movement or reaction in the other component in a causal manner.

Catheter 40 with its corresponding magnetic coupler 20 mounted thereon, may be operatively connected to the distal end 62 of a surgical device 60 or may be passed through a port 66 of a surgical device to extend from the distal end 62 thereof, as shown in FIG. 4.

Each device 52, 60 is deployed in the patient, through a natural orifice, through a small keyhole incision, or a larger incision made in open surgery, and directed towards, although a distance from, each other. The catheters 30, 40 are extended toward each other within the patient to place the magnetic couplers 10, 20, blindly or with the aid of a radiological image, in the same approximate vicinity of one another until the opposite magnetic ends 12, 22 attract, as shown in FIG. 2 and FIG. 4. When attracted to each other, couplers 10, 20 align and couple sufficiently to enable accurate and confident passage of an instrument 50, such as a needle, guide wire, and/or another catheter through the channels formed by the aligned catheters 30,40 and the pair of couplers 10, 20. The instrument 50 may be rigid or flexible.

As shown in FIG. 2, where the nature of the procedure requires one or more layers of tissue 100 between the two catheters 30, 40 to be punctured or an incision made, the magnetic attraction between the two magnetic couplers 10, 20, will align the couplers and allow a needle or blade to be passed through one catheter to the tissue layer or layers 100 and, following the puncture or incision of the tissue 100, through the tissue 100 to the aligned channel of the other catheter. Radio frequency current may be carried through an instrument 50, for example a wire, or through one or both catheters 30, 40 for use in forming a passage through tissue 100 when the needle or blade alone can not pierce the tissue. The instrument 50 in this embodiment is operatively connected to a source of RF energy which may be activated at the discretion of the clinician during a given procedure. Any suitable known source of RF energy, such as monopolar RF energy, may be passed through the wire, for example, to the tissue to assist in piercing the tissue. Those skilled in the art will appreciate that the patient lies on any suitable known grounding pad when RF current is used.

In still other procedures, no layers of tissue, tissue bands, or tissue walls will block or otherwise interfere with the coupling of coupler 10, 20, so the couplers 10, 20 will join and form a continuous channel from one catheter to the other catheter without having to pierce tissue. In either kind of procedure, the instrument 50 is thereby able to be passed through one surgical device 52 or 60 directly into the channel 66 or 54, respectively, of the opposing surgical device 60 or 52.

The instrument 50 can be left in place for accurate placement of other devices such as tubular magnets, which can be slid over the wires from both starting points (e.g., peritoneal and extracorporeal via the mouth) and then put into their desired position. The foregoing procedure can facilitate a magnetic compression anastomosis.

The embodiments of the devices described herein may be introduced inside a patient using minimally invasive or open surgical techniques. Endoscopic minimally invasive surgical and diagnostic medical procedures are used to evaluate and treat internal organs by inserting a small tube into the body. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, anus, and/or vagina, for example. Some portions of the devices may be introduced to the tissue treatment region percutaneously or through small—keyhole—incisions.

A flexible endoscope may be introduced either through a natural body opening or via a trocar through a relatively small—keyhole—incision (usually 0.5-2.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An alignment and guidance system comprising:
a first coupler having a free magnetic end defining a first magnetic field around the free end of the first coupler, the first coupler having an attachable end and defining a channel therethrough, the attachable end being structured for attachment to an open end of a first catheter; and,
a second coupler having a free magnetic end defining a second magnetic field around the free end of the second coupler, the second coupler having an attachable end and defining a channel therethrough, the attachable end being structured for attachment to an open end of a second catheter and the free magnetic end of the second coupler being magnetically attracted to the free magnetic end of the first coupler such that upon contact between the first and second magnetic fields, the first and second free ends align to align the first and second channels and the open ends of the first and second catheters.

2. The alignment and guidance system recited in claim 1 wherein the attachable end of the first coupler is releasably attached to the first catheter and the attachable end of the second coupler is releasably attached to the second catheter.

3. The alignment and guidance system recited in claim 2 wherein the first catheter is operatively connected to a surgical instrument.

4. The alignment and guidance system recited in claim 2 wherein the second catheter is operatively connected to a surgical instrument.

5. The alignment and guidance system recited in claim 2 wherein each of the first and second catheters defines a channel and upon the contact in use between the first and second magnetic fields, the first and second free ends of the couplers couple to define a continuous channel between the first and the second catheters.

6. The alignment and guidance system recited in claim 1 wherein the attachable end of the first coupler is fixed to the first catheter and the attachable end of the second coupler is fixed to the second catheter.

7. The alignment and guidance system recited in claim 2 wherein each of the first and second catheters defines a channel, the system further comprising an instrument for passage through the channel of one of the first or second catheters.

8. The alignment and guidance system recited in claim 7 wherein the instrument carries a radio frequency current for use in forming a passage through tissue.

9. The alignment and guidance system recited in claim 1 wherein the free magnetic ends of each of the first and second couplers are made of a biocompatible ferromagnetic material.

10. The alignment and guidance system recited in claim 1 wherein the first and second couplers are made of a biocompatible ferromagnetic material.

11. A method of aligning channels of two catheters in an internal site within a patient during a therapeutic, surgical or diagnostic procedure comprising:
attaching a coupler to a free end of each of two catheters, wherein each coupler has an end attached to a different one of the catheters and a free magnetic end defining a magnetic field around said free end, wherein each coupler defines a channel therethrough from the attached to the free end;
directing one catheter from a first entry point of a patient to an internal site within the patient with the free magnetic end of the coupler positioned at the leading distal end of said one catheter;
directing the other catheter from a second entry point of the patient to the internal site within the patient with the free end positioned at the leading distal end of said other catheter; and,
bringing the magnetic fields of the free ends of each coupler into contact to thereby attract the free ends of each coupler to the other and align the channels of each coupler.

12. The method recited in claim 11 wherein at least one catheter defines a channel therethrough and the method further comprises inserting an instrument through the channel of said one catheter through the free end of the coupler attached thereto toward the free end of the other coupler.

13. The method recited in claim 12 wherein the instrument is a guide wire.

14. The method recited in claim 12 wherein one or more layers of tissue separate the first and second couplers, the method further comprising applying a radio frequency current to the tissue to form a passage through the tissue.

15. The method recited in claim 14 wherein the method further comprises coupling the free ends of the couples to define a continuous passage through the channels of each catheter and each coupler.

16. The method recited in claim 12 wherein one or more layers of tissue separate the first and second couplers, and wherein the instrument is a blade, the method further comprising making an incision in tissue separating the free ends of the first and second couplers.

17. The method recited in claim 16 wherein making an incision comprises applying a radio frequency current to the tissue to form a passage through the tissue prior to cutting the tissue with the blade.

18. The method recited in claim 16 wherein the method further comprises coupling the free ends of the couples to define a continuous passage through the channels of each catheter and each coupler.

19. The method recited in claim 18 wherein the method further comprises, withdrawing the blade from the channel of said one catheter and passing a second instrument through the channel of one of the catheters through the continuous passage.

20. The method recited in claim 11 wherein each catheter defines a channel therethrough and the method further comprises inserting an instrument through the channel of one catheter through the free end of the coupler attached thereto into the free end of the other coupler and into the channel of the catheter to which the other coupler is attached.
